(19) United States
(12) Patent Application Publication
(10) Pub. No.: US 2006/0198942 A1
(43) Pub. Date: Sep. 7, 2006

(54) System and method for coating a medical appliance utilizing a vibrating mesh nebulizer

(76) Inventors: Timothy O'Connor, Claregalway (IE); Niall Grenham, Oranmore (IE); David McMorrow, Galway City (IE); James Shannon, Galway (IE); Travis Schauer, Delano, MN (US); Thomas Holly, Athenry (IE)

Correspondence Address: KENYON & KENYON LLP, ONE BROADWAY, NEW YORK, NY, 10004, US

(21) Appl. No.: 11/326,744

(22) Filed: Jan. 5, 2006

Related U.S. Application Data
(63) Continuation-in-part of application No. 11/073,197, filed on Mar. 4, 2005.
(63) Continuation-in-part of application No. 11/073,198, filed on Mar. 4, 2005.

(51) Int. Cl. A61L 33/00

(52) U.S. Cl. 427/2.1

(57) ABSTRACT

A method and device for coating a medical device, such as a stent, including forming coating droplets using a mesh nebulizer and transporting the coating droplets to the medical device, for example through a converging chamber. The coating droplets may be accelerated to a speed sufficient to break the coating droplets into smaller droplets upon impact with the medical device. The mesh nebulizer may have a convex inlet side and may form a converging plume of coating droplets. The mesh nebulizer may have one or more groups of pores, the pores within each group may be subject to similar amplitudes of vibration. The coating material may be heated or cooled prior to nebulizing. The chamber may include baffles configured to allow only a predetermined size range of coating particles to pass.

### RELATED APPLICATIONS

This application is a continuation-in-part of U.S. patent application Ser. No. 11/073,197, filed Mar. 4, 2005, and entitled, “Method Of Producing Particles Utilizing A Vibrating Mesh Nebulizer For Coating A Medical Appliance, A System For Producing Particles, And A Medical Appliance,” and of U.S. patent application Ser. No. 11/073,198, filed Mar. 4, 2005, and entitled, “Method Of Coating A Medical Appliance Utilizing A Vibrating Mesh Nebulizer, A System For Coating A Medical Appliance, And A Medical Appliance Produced By The Method,” both of which are hereby incorporated by reference in their entirety.

### FIELD OF THE INVENTION

The present invention relates to medical devices. More particularly, the present invention relates to a method of coating a medical device using a vibrating mesh nebulizer to produce a mist of coating material, a system for coating a medical device, and a medical device produced by the method.

### BACKGROUND INFORMATION

Medical devices may be coated so that the surfaces of such devices have desired properties or effects. For example, it may be useful to coat medical devices to provide for the localized delivery of therapeutic agents to target locations within the body, such as to treat localized disease (e.g., heart disease) or occluded body lumens. Localized drug delivery may avoid some of the problems of systemic drug administration, which may be accompanied by unwanted effects on parts of the body which are not to be treated. Additionally, treatment of the afflicted part of the body may require a high concentration of therapeutic agent that may not be achievable by systemic administration. Localized drug delivery may be achieved, for example, by coating balloon catheters, stents and the like with the therapeutic agent to be locally delivered. The coating on medical devices may provide for controlled release, which may include long-term or sustained release, of a bioactive material.

Aside from facilitating localized drug delivery, medical devices may be coated with materials to provide beneficial surface properties. For example, medical devices are often coated with radiopaque materials to allow for fluoroscopic visualization while placed in the body. It is also useful to coat certain devices to achieve enhanced biocompatibility and to improve surface properties such as lubriciousness.

Metal stents may be coated with a polymeric coating that may contain a dissolved and/or suspended bioactive agent. The bioactive agent and the polymeric coating may be dissolved in a solvent mix and spray coated onto the stents. The solvent may then evaporate to leave a dry coating on the stent.

Conventional spray-coating technology may require pressurized gas in order to produce a spray plume. This may result in a very high velocity spray plume. Because of the high velocity spray plume, long distances between a spray nozzle and a stent may be used in order to deliver a good coating finish. This may result in poor material efficiency, sometimes on the order of 1%. Furthermore the use of pressurized gas may increase manufacturing costs.

Webbing may be a problem with two-fluid gas atomisers, particularly when coating large vessel coronary stents.

In the manufacture of a drug eluting stent, there are a number of challenges. Goals in the manufacture of coating stents include precise coating weight and complete encapsulation of stent struts, with minimal webbing between struts. Additionally, a stent may preferably be coated with a uniform coating on the inside and the outside of the stent and may be required to meet a product specification for kinetic drug release (KDR).

Medical devices may be coated using spray technology. This may entail the use of a two-fluid atomiser, or spray nozzle. The atomizer may be supplied with coating solution and nitrogen gas. The nozzle may be configured so that the coating solution forms a thin film on the pre-filming face of the nozzle, and droplets may then be sheared off the film by the flow of atomising gas.

Spray coating may have a number of limitations. In a spray coating operation, droplet size and droplet velocity may be inextricably linked. It may not be possible to control either of these factors without impacting the other. Additionally, droplet size may only be controlled within a relatively large window due to the gas atomization process. Atomization energy is provided by the nitrogen gas stream. This may result in a very high velocity with a correspondingly high energy spray plume, which is a significant contributor to difficulty in fixturing stents during the coating process.

Droplet size may be a critical factor in controlling kinetic drug release. Precise control of droplet size may be important in order to develop a high degree of control of kinetic drug release.

Furthermore, it has been shown that the high velocity spray plume produced by two-fluid atomisers may cause stents to get blown out of alignment on the stent coating fixtures. This has led to difficulty in controlling coat weight, and has led to coating bare spots due to uncontrolled interaction between a stent and a coating fixture. One approach to counter this issue has been to significantly increase the nozzle-to-stent distance. While this reduces the movement of the stent on the coating fixture, it may result in low coating material efficiencies, perhaps on the order of 1%. A further disadvantage of two-fluid atomisers is that many of the droplets may bounce off the object to be coated, which may further limit the material efficiency. The coating of flexible, self-expanding stents and/or longer stents may create a further difficulty whereby the stent is moved, flexed and/or bent on a fixture during coating. There is therefore a need for reducing coating defects in medical devices.

### BRIEF DESCRIPTION OF THE DRAWINGS

FIG. 1 is a schematic diagram of an exemplary system according to the present invention.

FIG. 2 is a zoomed-in view of an exemplary embodiment of a nebulizer.

FIG. 3 illustrates an exemplary embodiment of the present invention including a coating chamber.

FIG. 4 is a schematic diagram of an exemplary embodiment of a nebulizer.

FIG. 5 is another schematic diagram of another exemplary embodiment of a nebulizer.

FIG. 6 is a flowchart illustrating an exemplary method according to the present invention.

FIG. 7 is a schematic diagram of another exemplary embodiment of the present invention.

FIG. 8 is a schematic diagram of another exemplary embodiment of the present invention.

FIG. 9 is a perspective view of the insert shown in cross section in FIG. 8.

FIG. 10 is a schematic diagram of another exemplary embodiment of the present invention.

FIG. 11A is a schematic diagram of an exemplary embodiment of the mesh nebulizer having centered pores.

FIG. 11B is a schematic diagram of an exemplary embodiment of the mesh nebulizer having a ring of pores with consistent amplitudes of vibration.

FIG. 11C is a schematic diagram of an exemplary embodiment of the mesh nebulizer having a double ring of pores with consistent amplitudes of vibration.

FIG. 11D is a schematic diagram of an exemplary embodiment of the mesh nebulizer have a plurality of pore groups with consistent amplitudes of vibration arranged in a ring.

FIG. 11E is a schematic diagram of an exemplary embodiment of the mesh nebulizer having a strip of pores with consistent amplitudes of vibration.

### DETAILED DESCRIPTION

A method of coating a medical device is provided that includes contacting a coating material with a first side of a mesh nebulizer. The mesh nebulizer includes at least one aperture. The method also includes vibrating the mesh nebulizer and arranging the medical device in a region of a second side of the mesh nebulizer. The second side is opposite the first side.

The mesh nebulizer may form droplets of the coating material.

The method may include transporting the droplets from the mesh nebulizer to the medical device. The transporting may be performed by a gas source. The transporting may be performed by gravity, and the mesh nebulizer may be positioned above the medical device.

The method may include providing an electrostatic potential between the mesh nebulizer and the medical device. The mesh nebulizer may impart an electrostatic charge to the droplets of the coating material, as detailed in the disclosure of U.S. patent application Ser. No. 10/744,483, filed Feb. 10, 2004, and entitled, “Apparatus and Method for Electrostatic Spray Coating of Medical Devices,” herein incorporated by reference in its entirety and assigned to assignee of the current patent application.

The method may include selecting a size of the at least one aperture of the mesh nebulizer. The size of the apertures may determine the size of the droplets. The size of the at least one aperture may be between about 0.1 μm and about 200 μm, may be between about 3 μm and about 20 μm, and may in particular be about 10 μm.

The method may include selecting a frequency of the vibration of the mesh nebulizer. The method may include varying the frequency of the vibration of the mesh nebulizer. The method may include selecting an amplitude of the vibration of the mesh nebulizer. The method may include varying the amplitude of the vibration of the mesh nebulizer.

The coating material may include at least one of a protein and a peptide.

The method may include selecting a location of the at least one aperture on the mesh nebulizer and/or a quantity of the at least one aperture on the mesh nebulizer.

The method may include fixturing the medical device to allow the coating material to contact about all of a surface of the medical device.

A medical device is provided having a coating applied by a method. The method includes contacting a coating material with a first side of a mesh nebulizer. The mesh nebulizer includes at least one aperture. The method also includes vibrating the mesh nebulizer and arranging the medical device in a region of a second side of the mesh nebulizer. The second side is opposite the first side.

A system is provided for coating a medical device that includes a coating source, a mesh nebulizer, an arrangement for vibrating the mesh nebulizer, and an arrangement for holding the medical device.

A method is provided of coating a medical device that includes directing at least two small aperture tubes at a collision region and forcing a coating material out of the apertures of the tubes. The method also includes arranging the medical device in another region adjacent to the collision region.

An exemplary embodiment of the present invention proposes the use of nebulizer technology in the coating of medical devices, in particular drug eluting stents. Nebulizers are medical devices used to vaporise medications for inhalation, specifically to convert liquid drugs into fine droplets for inhalation. Small, controllable droplet size, with typical size ranges in the order 1 to 5 microns, may be achievable with a nebulizer. A low energy droplet cloud may be desirable and therefore converting a solution into small droplets without imparting high velocities to the droplets may be desired. Additionally precise control of a delivered drug volume may be desirable.

A component of some nebulizer designs is a convex mesh which may have numerous, precisely-sized holes. The drug to be administered may be placed in the concave side of the mesh, and the mesh may be vibrated at high frequency using a piezoelectric drive. This may result in the drug being converted into a cloud of small droplets, which may be delivered on the lower (convex) side of the mesh.

Use of nebulizers instead of two-fluid atomisers may offer several advantages in coating drug eluting stents, or any other medical device. Extremely precise droplet size may be possible with a nebulizer. Precise droplet size control may be advantageous since it has been demonstrated that droplet size correlates directly to kinetic drug release. Precise control of kinetic drug release may be achievable with precise control of droplet size. Additionally, droplet size may be programmable. In particular, geometric changes may be made to the nebulizer to provide a specific desired droplet size. Additionally, droplet size may be controlled independently of droplet velocity. Due to the low velocity of the plume coupled with fine droplet size, very small stent features may be coated without webbing. No atomisation gas may be required.

Use of this method of atomisation may offer several advantages. The size of the droplets may be extremely precise because it may be determined by the size of the holes in the mesh (which may be tailor-made to suit the application). This may contribute to precise control of kinetic drug release and an ability to coat complex geometries with small feature dimensions. Due to the absence of atomisation gas, the droplets may fall away from the mesh under the force of gravity at low velocity. The volume of liquid atomised, and the droplet velocity, can also be precisely controlled by adjusting the frequency and amplitude of the mesh vibration. Furthermore, the number of holes in the mesh and their layout on the mesh can be tailored. This could enable greatly increased coating material efficiency, as the atomised cloud could be sized to suit the stent being coated. Furthermore, fixturing of stents during the coating process can be greatly simplified, as there is no longer a need to hold the stent securely to prevent it getting blown away by the atomisation gas. This may be particularly important for future generation stents which may be longer and more easily damaged during handling.

In an alternative exemplary embodiment, an electrostatic system may be integrated with the nebulizer. This may enable higher material efficiency while retaining precise droplet size. No atomisation gas may be required in the exemplary method, and consequently stent fixturing may be greatly simplified. Therefore, the coating process may be well controlled. An electrostatic system may be accomplished by attaching a power source to the nebulizer mesh and providing a grounding contact to the stent. This may deliver higher material efficiency.

An alternative nebulizer design may atomise fluids using two capillary tubes, which may be oriented at an angle to each other. The fluid to be atomised may be pumped through the tubes. Small droplets may exit the ends of the tubes, and the size of these droplets may be determined by the diameter of the tube. Due to the angular arrangement, the droplets from each tube may collide, leading to further break-up of the droplets. The droplet size produced by this type of nebulizer may be approximately 5 microns. A nebulizer using two capillary tubes in angular arrangement may be configured in a number of ways. In particular, capilliary tube size, diameter, angle, fluid flow rate are key parameters.

Since nebulizers may not require a propellant gas, there may be fewer factors controlling the aerosol properties. However, the aerosol plume may require a gas current to entrain the plume so that it flows in the direction of the stent. This gas flow may be directed and accelerated towards the stent by means of a venturi type baffle arrangement.

A nebulizer may be configured in a number of ways to facilitate stent coating. In particular, mesh hole size, location and quantity may be altered. Vibration frequency and amplitude may also be tailored. Materials may be changed to facilitate use with solvent-based coatings.

The stent may be rotated and/or moved axially, or alternatively may remain fixed, depending on the size of the atomised cloud. Stent fixturing may be accomplished by supporting the stent on a pair of wires, possibly without the need to pass a wire through the center of the stent. This may accelerate the stent fixturing process, and substantially improve the quality of the stent coating, particularly on the stent internal surface. Furthermore, this method may enable the coating of more delicate stents with increasingly complex feature details.

The design of the nebulizer may facilitate the delivery of more than one fluid to the rear surface of the mesh, thus enabling coat mixing at the point of application. This may offer benefits where short shelf-life materials are used in coating, or in the use of coating materials which are not suitable for long-term storage when pre-mixed. This approach may also be used to alter coat composition during the application of coating, thus enabling creation of products where kinetic drug release or coat composition can be altered for different areas of the product being coated. Arrays of pores may be designed in various shapes, including rectangles and lines. Pores may be of different sizes to accommodate different materials and may be separated on the concave side of the nebulizer by walls or other barriers. Different materials may mix in the plume after being nebulized through different sized pores.

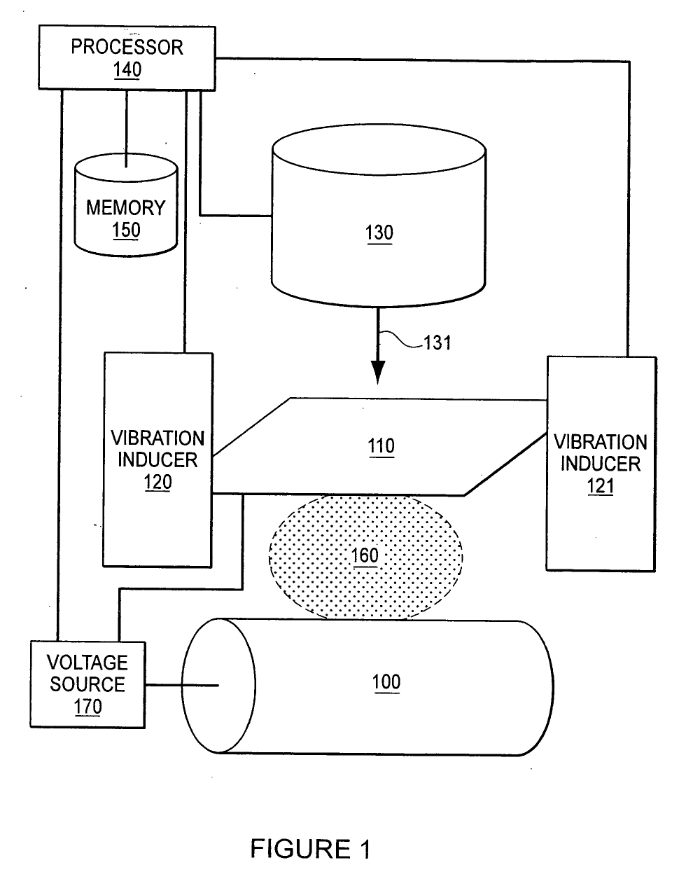

FIG. 1 is a schematic diagram of an exemplary system according to the present invention. Stent 100 is shown positioned below nebulizer mesh 110. Nebulizer mesh 110 is positioned between vibration inducers 120, 121. Alternatively, there may be more or fewer vibration inducers 120, 121. Vibration inducers 120, 121 may induce vibration in a direction parallel and/or perpendicular to nebulizer mesh 110, and may induce a complex vibration. Nebulizer mesh 110 includes one or more pores that may be between about 0.1 μm and about 200 μm, may be between about 3 μm and about 20 μm, and may be about 10 μm. The pores in nebulizer mesh 110 may be of uniform size or may be variably sized. Additionally, the pores in nebulizer mesh 110 may be frustoconical, vortex-shaped, and/or any other appropriate shape. Coating source 130 provides a coating material in the direction of arrow 131 to nebulizer mesh 110. After passing through the pores of nebulizer mesh 110, the coating material may form plume 160, which may consist of droplets. Droplets having a diameter of about 5 microns may be produced, for example, by a pore size of 3 microns in nebulizer mesh 110. The droplets in plume 160 may have a very narrow size distribution, and therefore may produce a uniform coating on stent 100. Processor 140 coupled to memory 150 may contain and/or execute instructions for operating coating source 130, vibration inducers 120, 121, and/or voltage source 170. Voltage source 170 may be connected to stent 100 and/or nebulizer mesh 110 and may impart an electric potential that provides a charge to the droplets in plume 160 that is opposite to the charge on stent 100. Plume 160 may be directed to coat stent 100 by gravity, by an additional gas source, and/or by an electrostatic potential.

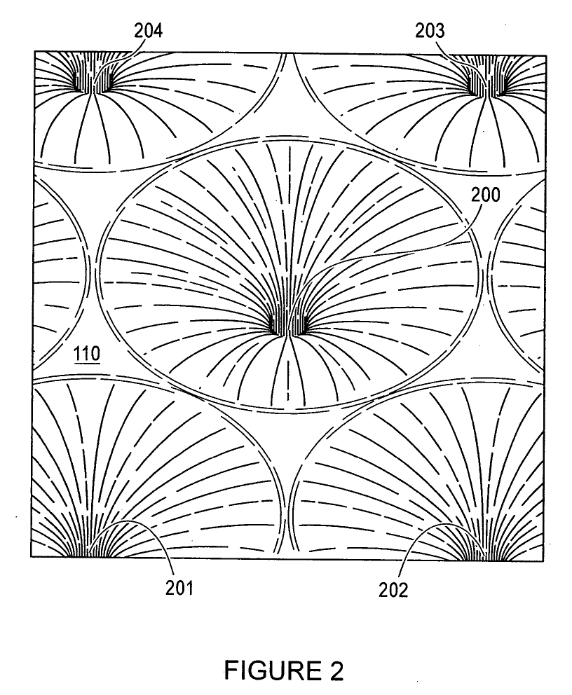

FIG. 2 is a zoomed-in view of an exemplary embodiment of nebulizer mesh 110. Nebulizer mesh 110 includes pores 200, 201, 202, 203, 204, which in this exemplary embodiment are vortex-shaped. Alternatively, pores 200, 201, 202, 203, 204 of nebulizer mesh 110 may be frusto-conical or any other appropriate shape.

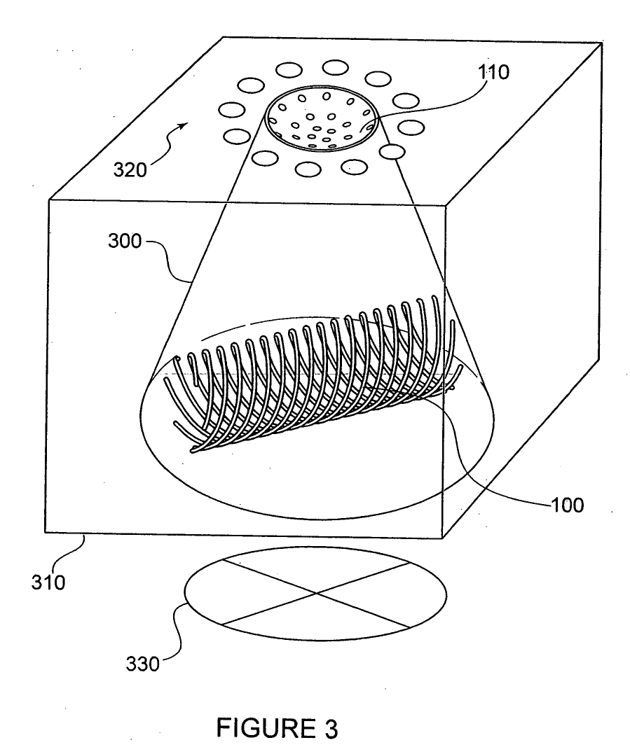

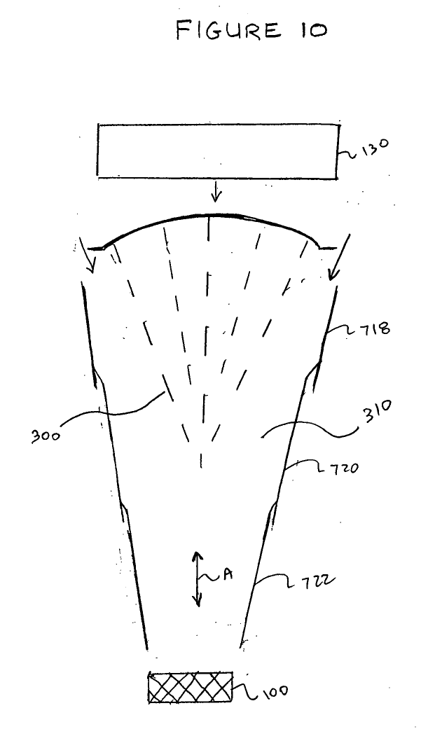

FIG. 3 illustrates an exemplary embodiment of the present invention including coating chamber 310. Nebulizer mesh 110 is situated at an upper portion of coating chamber 310. Coating chamber 310 encloses stent 100. Coating chamber 310 includes gas intakes 320, which may allow a gas to enter coating chamber 310. Gas intakes 320 may also provide a flow of gas under pressure to coating chamber 320. Gas exhaust 330 may remove gas and or excess material (for instance, coating material that has not adhered to stent 100) from coating chamber 320. Alternatively, coating chamber 310 may be airtight and/or evacuated, or may enclose an inert gas. When a coating material is arranged on mesh nebulizer 110, and mesh nebulizer 110 is vibrated, cone plume 300 of coating material in coating chamber 310 may be formed. Cone plume 300 may settle on stent 100 arranged in cone plume 300 by gravity, or may be assisted in moving toward stent 100 by a gas flowing from gas intakes 320 to gas exhaust 330. As detailed below with respect to FIG. 8, coating chamber 310 may have a venturi like baffle arrangement. The coating chamber 310 may decrease in cross section so as to accelerate the gas and coating material entrained in the gas towards the stent 100.

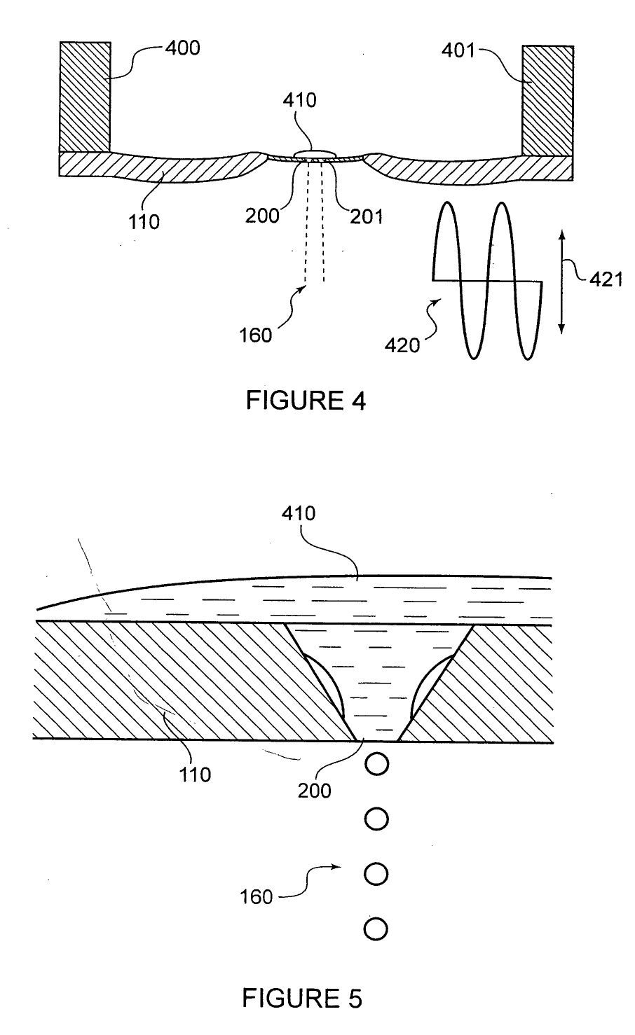

FIG. 4 is a schematic diagram of an exemplary embodiment of mesh nebulizer 110. Mesh nebulizer 110 includes pores 200, 201 and lateral barriers 400, 401. Alternatively, there may be more or fewer pores 200, 201, and/or more or fewer lateral barriers 400, 401. Coating material 410 is situated on a top side of mesh nebulizer 110, and is situated in a vicinity of pores 200, 201. Lateral barriers 400, 401 and/or another element may impart a vibration to mesh nebulizer. The vibration may correspond to sinusoid 420, and may consist of a vibration in a direction of double arrow 421. Alternatively or additionally, a lateral vibration in a plane of nebulizer mesh 110 may be induced. The vibration of nebulizer mesh 110 may induce coating material 410 to pass through pores 200, 201 to create plume 160.

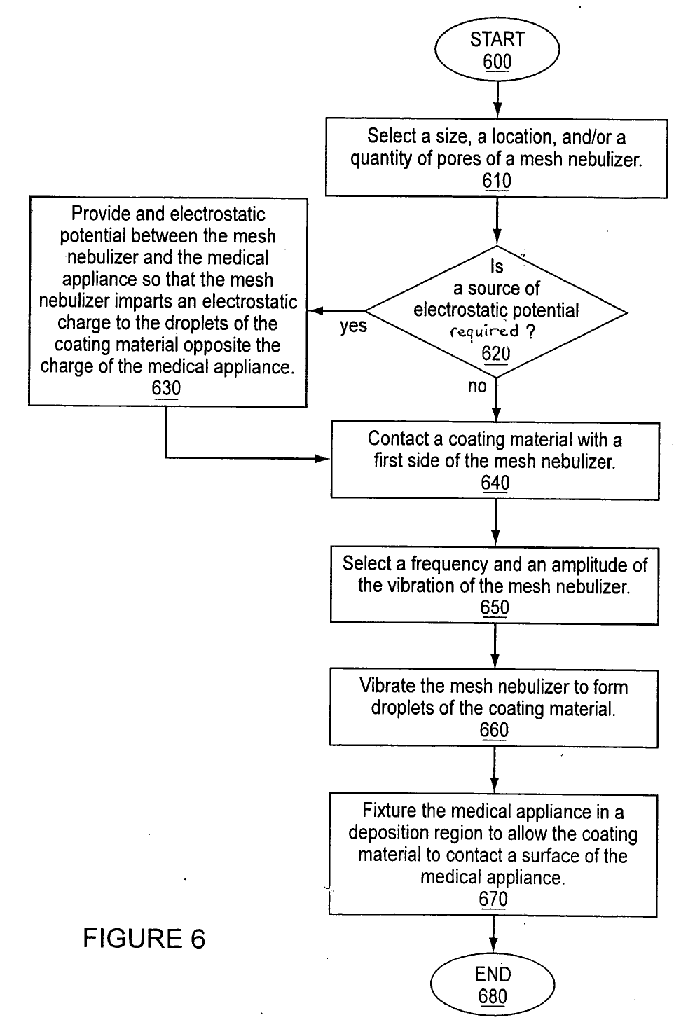

FIG. 5 is another schematic diagram of another exemplary embodiment of nebulizer mesh 110 showing a zoomed in view of pore 200. Pore 200 is frustoconical, though alternative shapes may be possible. Coating material 410 flows through pore 200 when nebulizer mesh 110 is vibrated to form plume 160, which may be composed of droplets of a small diameter. The droplets of plume 160 may have a narrow size distribution, and may be between about 0.1 μm and about 200 μm, or may be between about 3 μm and about 20 μm. In one exemplary embodiment, pore 200 may be about 3 microns in diameter and the droplets in plume 160 may be about 5 microns in diameter.

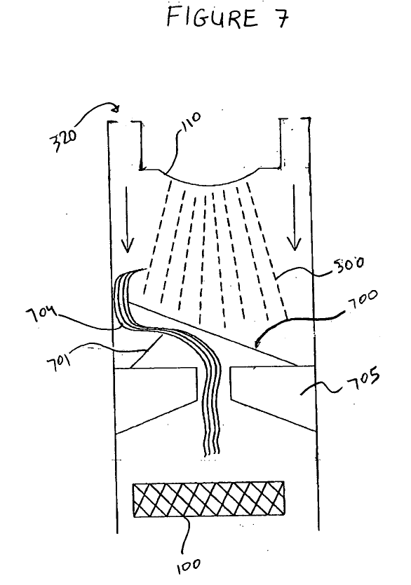

FIG. 6 is a flowchart illustrating an exemplary method according to the present invention. The flow in FIG. 6 starts in start circle 600 and proceeds to action 610, which indicates to select a size, a location, and/or a quantity of pores of a mesh nebulizer. From action 610, the flow proceeds to decision 620, which asks whether a source of electrostatic potential is required. If the response to decision 620 is affirmative, the flow proceeds to action 630, which indicates to provide an electrostatic potential between the mesh nebulizer and the medical device so that the mesh nebulizer imparts an electrostatic charge to the droplets of the coating material opposite the charge of the medical device. From action 630, the flow proceeds to action 640, which indicates to contact a coating material with a first side of the mesh nebulizer. From action 640, the flow proceeds to action 650, which indicates to select a frequency and an amplitude of the vibration of the mesh nebulizer. From action 650, the flow proceeds to action 660, which indicates to vibrate the mesh nebulizer to form droplets of the coating material. From action 660, the flow proceeds to action 670, which indicates to fixture the medical device in a deposition region to allow the coating material to contact a surface of the medical device. From action 670, the flow proceeds to end circle 680. If the response to decision 620 is negative, the flow proceeds to action 640.

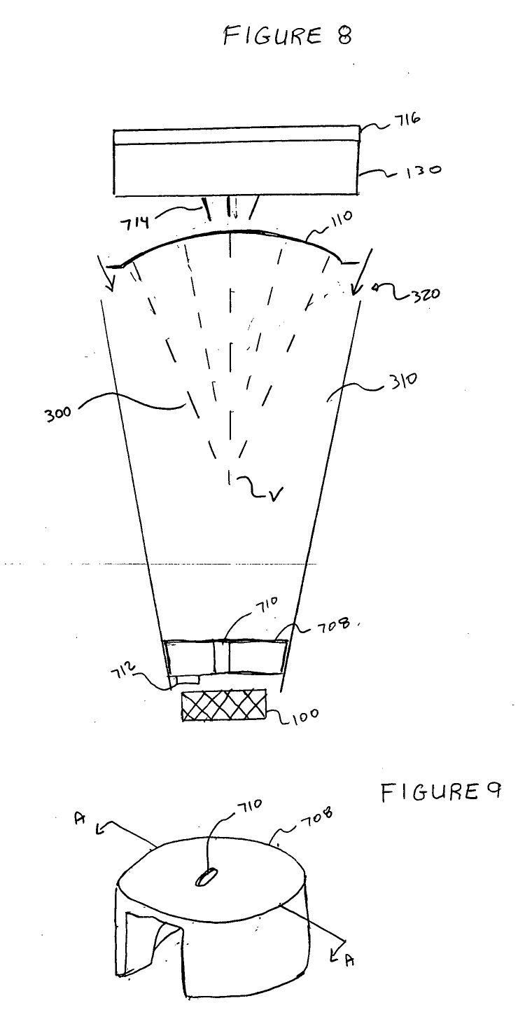

FIG. 7 illustrates an exemplary embodiment of the present invention including baffles 700, 701 used to reduce the droplet size distribution. Coating material arranged on mesh nebulizer 110 is nebulized and forms a plume 300 of coating droplets. The coating droplets are entrained in a gas stream 704 supplied via gas intake 320. Baffles 700, 701 require the gas stream 704 to make sudden changes in direction. Larger coating droplets entrained in the gas stream 704 unable to make the sharp turns impact the baffles 700, 701 and flow into one or more reservoirs 705 which may be tapped for reuse of the coating material. The coating drop size distribution may be controlled by changing the distance between baffles 700 and 701 and also by changing the angles of the baffles 700, 701 relative to the gas stream 704. The larger the angle of the baffles 700, 701, i.e., the more they approach horizontal, the smaller the resulting droplet sizes reaching the stent 100. A single baffle may be used or additional baffles may be added to further refine the droplet distribution.

FIG. 8 illustrates an exemplary embodiment of the present invention including a concave nebulizer mesh 110 leading into a coating chamber 310. The cross sectional area of the coating chamber 310 may decrease in a direction towards the stent 100 and, thus, may be used to accelerate a gas supplied via gas intake 320 and entrained coating droplets, exiting the nebulizer mesh 110, towards the stent 100. The gas supplied via gas intake 320 may include, for example, nitrogen, air, argon and/or carbon dioxide. The coating material may be accelerated to a speed at the stent 100 sufficient to overcome surface tension and, thus, break up at least some of the coating droplets into smaller droplets upon impact with the stent 100, which may be useful to build up the coating thickness on the stent 100. For example, the coating material may be accelerated to a speed between 1 and 100 meters per second or between 5 and 50 meters per second. In an exemplary embodiment of the present invention, a SIBs/THF/Toluene solution may be accelerated to 30 meters per second. The chamber 310 may be combined with an electrostatic power source to provide a charge to the droplets so as to increase their attraction to the stent 100.

The reduction in cross section of the chamber 310 may also by useful to reduce the size of the plume 300 so as to more closely match the size of the stent 100, which may increase coating material efficiency. The chamber 310 may, for example, be cone or funnel shaped or have cylindrical form with parallel walls. The chamber 310 may also have a traditional straight duct shape, as shown in FIG. 3. The nebulizer mesh 110 may have a concave configuration and may produce a converging cone shaped plume 300 which may be carried by the gas to the stent 100. Stent 100 may optionally be placed further upstream so as to lie at a vertex V of the plume 706, which is the point of highest droplet concentration. Positioning of the stent at the vertex V of the plume 300 may improve material transfer efficiency and reduce cycle time.

The velocity of the droplets exiting the chamber 310 may be controlled directly by adjusting the pressure differential and, correspondingly, the flow of gas to the stent 100. The pressure in the chamber 310 may adjusted to match or be higher or lower than atmospheric pressure. This enables control of the rate of evaporation of solvent from the droplets as they travel towards the stent 10, thus, enhancing control of the coating process and control of kinetic drug release. The size and shape of the plume 300 exiting the chamber 310 maybe controlled by adjusting the size of a downstream exit of the chamber 310. The size of the individual droplets may be controlled by adjusting the size of pores (not shown) on the nebulizer mesh 110. Use of the accelerating chamber 310 in combination with the nebulizer mesh 110 allows for independent control of droplet size and droplet velocity. As indicated above, in conventional spray nozzles, such as two fluid atomizers, droplet size and droplet velocity are inextricably linked and cannot be adjusted independently.

The volume of coating material delivered may be adjusted by altering the duty cycle of a vibration inducer, such as an electronic oscillator, used to drive the nebulizer mesh 110. The oscillator may be programmed so that it is not oscillating continuously, e.g., it is operated in a pulsed mode whereby the nebulizer mesh 110 is only driven for a percentage of the run time.

As indicated above, use of a concave nebulizer mesh 110 results in converging plume 300 which concentrates the plume 300 and, thus, results in a more material efficient coating system. The concave nebulizer mesh 110 forces the coating material to spread out over the convex outer surface which results in a utilization of a larger number of the pores used to create the plume 300. This is in contrast to the traditional nebulizer mesh where the coating material tends to pool or well-up on the outer surface of the nebulizer mesh.

One or more needles 714 may be used to supply coating material from one or more coating sources 130 containing one or more different coating materials. The coating materials may be supplied sequentially or simultaneously. Surfactants may be added to the coating material at the point of nebulization. Each needle 714 may be connected to a separate syringe pump. The different coating materials maybe delivered simultaneously or sequentially. Use of multiple coating materials will result in a plume containing droplets of various materials. The flow rate through each needle 714 may be regulated independently so as to control the droplet concentration within the plume 300 and, ultimately, the droplet concentration within a dried coating over the stent 100. The resulting droplet size for each coating material may be controlled independently by using coating materials of different viscosities, surface tensions and dielectric properties. A solvent may be used in the coating materials which may also be chosen so as to tailor the droplet size, mass and drying rate within the plume 300. Given that droplets accelerate at different rates depending on their mass, multiple layers of different materials can be sequentially built up on the stent 100 during one or more spray cycles. Further, coating materials with differing dielectric characteristics may be chosen so that each coating material accepts a different charge level which results in a preferential attraction of individual droplets within the plume 300 to a surface of the stent 100. The coating materials may be added at different temperatures to influence, for example, evaporation rates of the coating material. Further, solutions with different concentration of the same drug can be deposited simultaneously or sequentially to alter kinetic drug release.

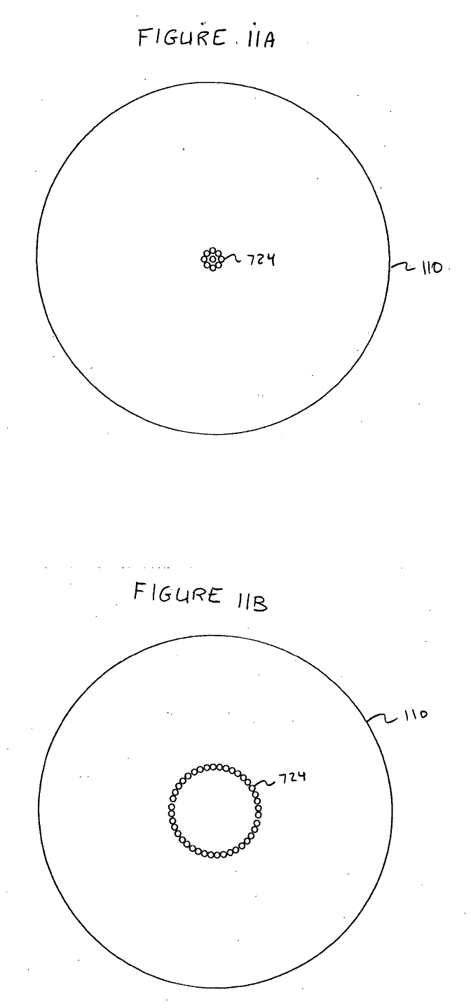

The gas stream may optionally pass through a hole 710 in an insert 708, which may be placed at the end of the chamber 310 just upstream of the stent 100. FIG. 9 is a perspective view of an exemplary embodiment of the insert, a cross section of which, taken along lines A-A, is included in FIG. 8 just above the stent 100. The shape of the hole 710 determines the shape of a plume of the coating material exiting the insert 708. Plumes of varying shape may be created by varying the shape of the opening 710. Although shown in the shape of a slot, hole 710 may have any shape. The opening 710 may be oriented perpendicular to the stent 100 so as to ensure that the plume is wider than any potential misalignment of the stent 100. The shape and size of the opening 710 may also be changed to control the gas flow, and hence the droplet velocity, for a given gas pressure. Further, the droplet density within the spray plume can also be altered by changing the cross sectional area of the opening 710. The geometry of the opening 710 may also be used to concentrate the spray plume directly over a small area of the stent 100 to maximize material efficiency. This becomes important when dealing with expensive materials such as bio-molecules and those used for gene therapy. The insert 708 may be made, for example, from stainless steel and heated by including a heating element 712 to give better control of in-flight droplet evaporation and drying. Electrodes (not shown) may also be placed in the opening 710 so as to charge the coating droplets as they pass through the opening 710. The stent 100 may be ground to accelerate the coating droplets towards it. The stent 100 may be rotated and/or moved linearly relative to the stream of coating droplets emerging from hole 710.

The coating materials may be cooled or heated using a temperature control unit 716, such as a cooler or heater. Cooling, for example, a solvent based coating material reduces the evaporation rate of the solvent and, thus, stabilizes the coating material's solids concentration. This allows the stent 100 to be coated with a less viscous solution, which may give a wetter surface finish. Heating, for example, a solvent based coating material increases the evaporation rate of the solvent and, thus, increases the coating material's solids concentration. This may provide for a dryer surface on the stent 100. Toluene and Tetrohydrofuran, for example, have reduced evaporation through cooling. Toluene and Tetrohydrofuran have flashpoints of 4.4 and −14 degrees Celsius, respectively. Cooling of the coating material allows for use of organic and non-organic solvents with low flashpoints. The temperature control unit 716 may comprise a tube, for example, made from aluminum, wrapped around the coating material source 130 or an inlet or outlet tube to the coating material source 130 through which a cooling or heating fluid is circulated.

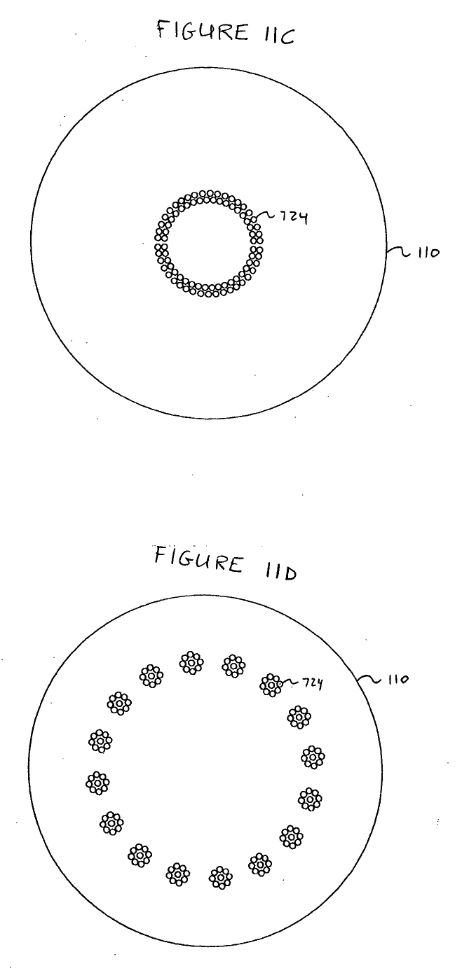

In an exemplary embodiment, the chamber 310 is defined by multiple cones 718, 720, 722 telescoped within one another, as illustrated in FIG. 10. This arrangement allows the cones to move along arrow A so as to control the distance between the chamber 310 and the stent 100. Chamber 310 may be defined by more or less than the three telescoping cones shown in FIG. 10.

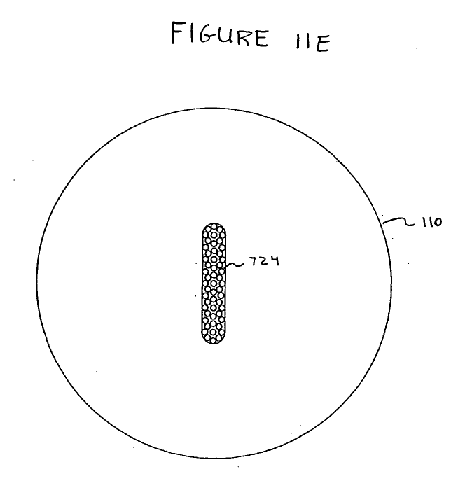

The amplitude of vibration may be non-uniform across the nebulizer mesh 110. This lack of uniformity results in an increase in the droplet size distribution, which may decrease material efficiency. In order to narrow the droplet size distribution, the nebulizer mesh 110 may limit the pores to those subject to similar amplitudes of vibration, for example, within 5% of each other. FIGS. 11A-11E show exemplary embodiments of the nebulizer mesh 110 with pores arranged in areas of consistent amplitude of vibration. FIG. 11A shows the nebulizer mesh 110 with pores 724 concentrated in the center. FIG. 11B shows the nebulizer mesh 110 with pores arranged in a ring towards the center. FIG. 11C shows the nebulizer mesh 110 with pores arranged in a double ring towards the center. FIG. 11D shows the nebulizer mesh 110 with circular groups of pores arranged in a ring shape. FIG. 11E shows the nebulizer mesh 110 with a strip of apertures.

Different coating materials can be introduced through different groups of pores across the nebulizer mesh 110 using, for example, multiple delivery pumps. The pore size can be tailored to the material being nebulized. Therefore, different groups of pores may have different diameters, each group being associated with a different solution for nebulizing. This results in specific droplet size distributions for the different coating materials. Apertures of various sizes can be mixed or located in various areas and, thereby, used to produce bi-modal or multi-modal droplet size distributions.

Further, tailoring the plume shape also may improve material efficiency. The number of pores in the nebulizer mesh 110 may be reduced from that of a standard vibrating disc nebulizer, e.g., which contains 1000 pores, to reduce the coating material flow rate while keeping the nebulizer mesh 110 wet thereby resulting in continuous droplet supply at an output side of the nebulizer mesh 110. The number of pores may be reduced to one so that a mono-dispersed droplet stream may be produced.

As used herein, the term “therapeutic agent” includes one or more “therapeutic agents” or “drugs”. The terms “therapeutic agents”, “active substance” and “drugs” are used interchangeably herein and include pharmaceutically active compounds, nucleic acids with and without carrier vectors such as lipids, compacting agents (such as histones), virus (such as adenovirus, andenoassociated virus, retrovirus, lentivirus and α-virus), polymers, hyaluronic acid, proteins, cells and the like, with or without targeting sequences.

The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; and any combinations and prodrugs of the above.

Exemplary biomolecules include peptides, polypeptides and proteins, including fusion proteins with molecular weights up to and above 200 kDa; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; anti-restenosis agents; and monoclonal antibodies. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, lipids and compounds have a molecular weight of less than 100 kD, inflammatory agents, and immune system modulators. A non-limiting example of an inflammatory agent is interleukin-1 and a non-limiting example of an immune system modulator is interferon beta-1a.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin-) cells including Lin-CD34−, Lin-CD34+, Lin-cKit+, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable. Non-limiting examples of suitable non-biodegradable polymers include polystrene; polyisobutylene copolymers and styrene-isobutylene-styrene block copolymers such as styrene-isobutylene-styrene tert-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent/therapeutic agent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The coating can be applied to the medical device by any known method in the art including dipping, spraying, rolling, brushing, electrostatic plating or spinning, vapor deposition, air spraying including atomized spray coating, and spray coating using an ultrasonic nozzle.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters, guide wires, balloons, filters (e.g., vena cava filters), stents, stent grafts, vascular grafts, intraluminal paving systems, implants and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

While the present invention has been described in connection with the foregoing representative embodiment, it should be readily apparent to those of ordinary skill in the art that the representative embodiment is exemplary in nature and is not to be construed as limiting the scope of protection for the invention as set forth in the appended claims.

What is claimed is:

1. A method of coating a medical device, comprising:
forming coating droplets using a mesh nebulizer;
transporting the coating droplets to the medical device at a speed sufficient to break at least some of the coating droplets into smaller droplets upon impact with the medical device.

2. The method of claim 1 wherein the coating droplets are transported via an accelerating gas stream.

3. The method of claim 1, wherein the coating droplets are formed by:
(a) contacting a coating material with a first side of a mesh nebulizer, the mesh nebulizer comprising at least one aperture; and
(b) vibrating the mesh nebulizer so as to produce the coating droplets.

4. A method of coating a medical device, comprising:
forming coating droplets using a mesh nebulizer; and
transporting the coating droplets to the medical device through a chamber having a cross section which reduces in size as it approaches the medical device.

5. The method of claim 4, further comprising controlling the rate of evaporation of solvent from the coating droplets by controlling the pressure in the chamber.

6. The method of claim 1, further comprising providing an electrostatic potential between the mesh nebulizer and the medical device, the mesh nebulizer imparting an electrostatic charge to the droplets of the coating material.

7. The method of claim 1, wherein the coating droplets pass through an opening in a plume control insert before reaching the medical device.

8. The method of claim 3, wherein the first side is contacted with a plurality of coating materials.

9. The method of claim 8, wherein the plurality of coating materials contact the first side simultaneously.

10. The method of claim 8, wherein the plurality of coating materials contact the first side sequentially.

11. The method of claim 8, wherein at least two of the plurality of coating materials have different masses, dielectric characteristics, viscosities, surface tension values or temperatures.

12. A method of coating a medical device, comprising:
forming coating droplets using a mesh nebulizer having one or more groups of pores, the pores within each group subject to similar amplitudes of vibration; and
transporting the coating droplets to the medical device.

13. The method of claim 12, wherein the pores at least one of (i) form a ring on the mesh nebulizer, (ii) form a strip passing through a center of the mesh nebulizer, and (iii) form a group concentrated in the center of the mesh nebulizer.

14. A method of coating a medical device, comprising:
one of cooling and heating a coating material so as to achieve a desired evaporation rate of the coating material;
forming coating droplets from the coating material using a mesh nebulizer; and
exposing the medical device to the coating droplets.

15. A method of coating a medical device, comprising:
forming a converging nebulized plume of coating droplets using a mesh nebulizer having one or more pores; and
directing the coating droplets towards the medical device.

16. The method of claim 15, wherein the converging nebulized plume of coating droplets is formed by contacting a coating material to a convex inlet side of the mesh nebulizer.

17. A method of coating a medical device, comprising:
forming coating droplets using a mesh nebulizer; and
transporting the coating droplets past one or more baffle plates to the medical device via a gas stream.

18. The method of claim 17, wherein the one or more baffle plates are configured so as to allow the coating droplets hitting the one or more baffles to flow back to a collection area.
